(19) United States
(12) Patent Application Publication
(10) Pub. No.: US 2022/0143295 A1
(43) Pub. Date: May 12, 2022

(54) PINCH VALVE MECHANISM

(71) Applicant: Multigate Medical Products Pty Ltd, Villawood NSW (AU)

(72) Inventors: Amy LEEDHAM, Villawood NSW (AU); Zongyun REN, Villawood NSW (AU); Joshua MCCARTHY, Villawood NSW (AU); Mark SPAILE, Villawood NSW (AU); Shirley LIU, Villawood NSW (AU)

(73) Assignee: Multigate Medical Products Pty Ltd, Villawood NSW (AU)

(21) Appl. No.: 17/430,923

(22) PCT Filed: Feb. 14, 2020
(86) PCT No.: PCT/AU2020/000016

§ 371 Date: Aug. 13, 2021

(30) Foreign Application Priority Data
Feb. 15, 2019 (AU) 2019100171
Feb. 15, 2019 (AU) 2019900491
Mar. 26, 2019 (AU) 2019901006

(51) Int. Cl. A61M 1/00; A61M 39/28

(52) U.S. Cl. CPC A61M 1/7415; A61M 39/28; A61M 1/774

(57) ABSTRACT

A pinch valve mechanism for use in a suction/irrigator device, wherein the device has a conduit for suction/irrigation. The pinch valve mechanism includes a first end having a first projection, a second end, operatively connected to the first end and a second projection. A spring means operatively connected to the second end, wherein the conduit is disposed between the first end and the second end such that the first projection and second projection are biased to pinch the conduit into a closed position. Compression of the spring means moves the first end away from the second end to move the conduit into an open position.

### TECHNICAL FIELD

The present invention relates to a pinch valve mechanism. In particular, the present invention relates to a pinch valve mechanism for a medical device, such as a suction/irrigator device.

### BACKGROUND OF THE INVENTION

The following references to and descriptions of prior proposals or products are not intended to be and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

During certain medical procedures, such as surgery or dental procedures, it is often necessary for a medical professional to provide irrigation fluid to a body site such as a wound or oral cavity. The irrigation fluid may be, for example, water, saline, or another biocompatible fluid. At the same time, it is often necessary to also apply suction to the body site in order to remove fluid and debris.

For example, during keyhole or laparoscopic surgery, it is often necessary to remove debris by suction and to irrigate the body site by delivering an irrigation fluid. In such procedures, the medical professional ensures that the suction forces are not overly strong so as to avoid tissue damage. When providing irrigation to the body site, it is also desirable to provide the irrigation fluid in a focused and accurate manner.

Typically, in a medical device, such as a suction/irrigation device a valve mechanism is used to close the suction tube for the irrigation function, or to close the irrigation tube, for the suction function. However, often, there is difficulty in achieving comfortable pressure for the button controlling the valve, especially given significant periods of use. Thus, the usability of the device can be affected with either too little or too much pressure on the button, thereby controlling the effectiveness of the suction/irrigation function.

The present invention seeks to provide a pinch valve mechanism which may ameliorate the foregoing shortcomings and disadvantages or which will at least provide a useful alternative, particularly for a suction/irrigation device, where controlled delivery during surgical procedures and a high level of precision is often required.

### SUMMARY OF THE INVENTION

According to one aspect of the invention, there is provided herein a pinch valve mechanism for use in a suction/irrigator device, the device having at least one conduit for suction/irrigation, the pinch valve mechanism including: a first end having a first projection; a second end, operatively connected to the first end and having a second projection; and, a spring means operatively connected to the second end, wherein the at least one conduit is disposed between the first end and the second end such that the first projection and second projection are biased to pinch the conduit into a closed position, and wherein, compression of the spring means moves the first end away from the second end to move the conduit into an open position.

According to one example, the spring means is compressed by a button. Thus, it will be appreciated that in a pre-use state, the at least one conduit is biased in a closed position, and will be opened by compression of a respective button. Thus, if there is more than one conduit, there may also be more than a respective button for closing/opening that particular conduit. It will further be appreciated that depression of the spring means is not limited to a button but can include any form of lever or actuating mechanism that is configured to compress the spring means.

According to another example, the usability of the button is affected by any one or a combination of variables including:
a. spring force;
b. shore hardness of the at least one conduit;
c. diameter of the at least one conduit; and,
d. thickness of the at least one conduit.

In yet a further example, the spring force has a range between about 5 and about 30 Newtons, the shore hardness of the at least one conduit is between about 30 and about 60 Shore A, the diameter of the conduit is between about 3 millimetres and about 15 millimetres, and/or the thickness of the conduit is between about 0.2 millimetres and about 4 millimetres.

According to another example, the first and second projections have respective first and second ends, where the first and second ends are formed in different shapes to compress the at least one conduit, the shapes including any one or a combination of: pointed in a V-shape; an inverse V-shape; curved or rounded or arcuate; and, flattened (and can be a combination of all shapes including peaks and troughs as required). Further, the first and second projections can be disposed to be substantially planar in respect of each other, or staggered.

According to yet a further example, a pressure exerted on the button is minimised by having a proportionally larger button surface area compared to valve area.

It will be appreciated by persons skilled in the art that any combination of the features described herein is possible.

### BRIEF DESCRIPTION OF THE DRAWINGS

The invention may be better understood from the following non-limiting description of a preferred embodiment, in which:

FIG. 1 is a perspective view of an example medical device, which can include the pinch valve mechanism as described herein;

FIG. 2 is a perspective open view of an example medical device including a pinch valve mechanism;

FIG. 3 is an open plan view of an example medical device including a pinch valve mechanism;

FIGS. 4 to 12 are side views of example pinch valve mechanisms, showing different shapes and types of projections of the pinch valve mechanism;

FIG. 13 shows an example of tube/conduit pressure forces that may apply as a result of the pinch valve mechanism described herein;

FIG. 14 shows an example valve pressure forces that may apply to a pinch valve mechanism as described herein; and,

FIG. 15 is a flow diagram showing an example of the interrelationship between the features of the pinch valve mechanism and the features which can affect the usability of the mechanism.

### DETAILED DESCRIPTION OF THE DRAWINGS

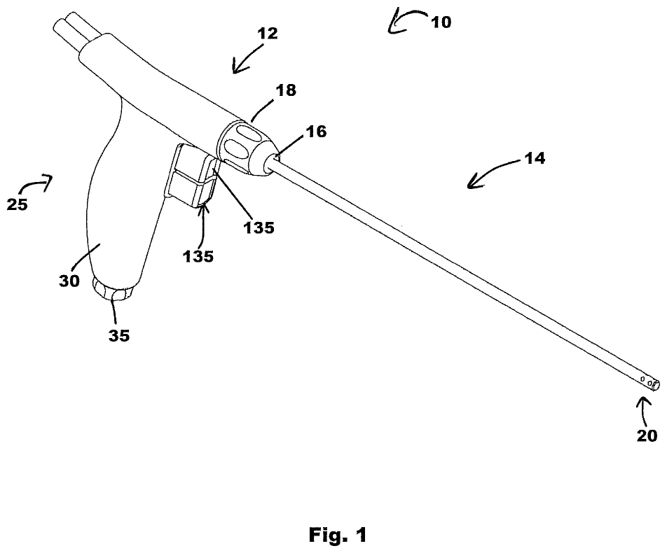

An example of a pinch valve mechanism 100 for a medical device 10 is shown in FIGS. 1 to 12.

It will be appreciated that the pinch valve mechanism 100 described herein can be used for any suitable suction/irrigation device, such as, for example, the Suction and Irrigation Apparatus as described in WO 2017/219070, and Australian patent no. 2019100171, the entire contents of both of which is incorporated herein by reference.

FIG. 1 shows an example of a medical device, a suction/irrigator device 10, which can provide suction/irrigation to a body site (not shown). In this example, the device 10 includes a body 12 and a shaft 14 attached thereto. The shaft 14 has a proximal end 16 for mounting to a first terminal 18 of the body 12, and a distal end 20 for juxtaposition with the body site.

The body 12 includes a housing 25 for covering various internal components of the device 10 as described in further detail below. The housing 25 includes a handle portion 30 to facilitate a user to hold and operate the apparatus 10. The body 12 includes a first terminal 18 and a second terminal 35 for selectively receiving the proximal end 16 of the shaft 14. The first terminal 18 is disposed generally perpendicularly to the second terminal 35.

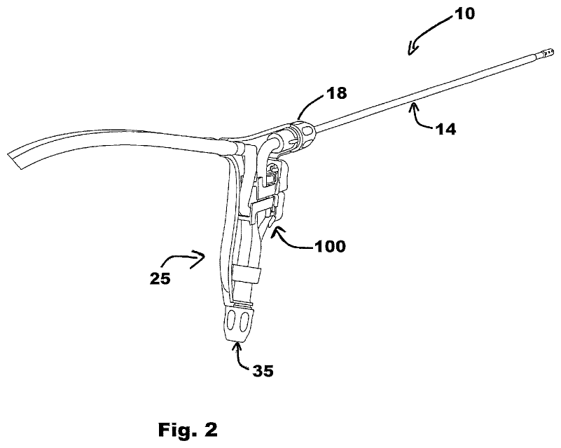

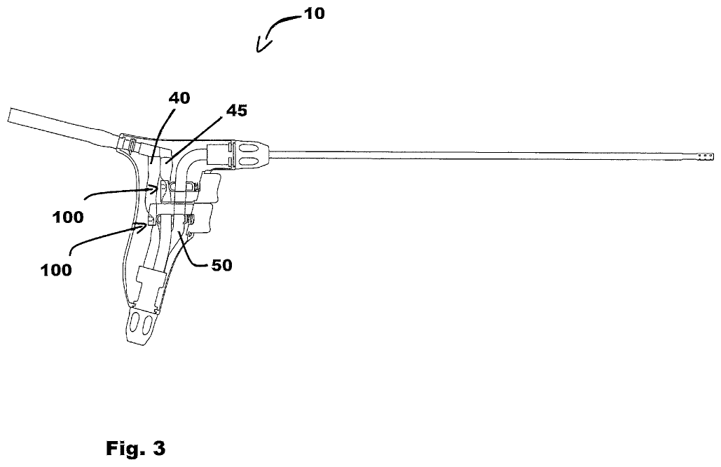

FIGS. 2 and 3 show the internal mechanism of the device 10. As shown in these examples, the device 10 includes an irrigation conduit 40 for delivering irrigation fluid to the shaft 14, and a suction conduit 45 for providing a negative pressure at the shaft 14 so that suction can be carried out at the body site. The apparatus 10 further includes an internal conduit 50 for connecting, and providing fluid communication between, the first and second terminals 18, 35. When fully assembled, the conduits 40, 45, and 50 are held in place by parts of the pinch valve mechanism 100.

The pinch valve mechanism is shown further in the examples of FIGS. 2 to 12. In these particular examples, there is provided a pinch valve mechanism 100 for use in a suction/irrigator device 10. As described, the device 10 has at least one conduit 40/45 for the suction/irrigation function.

As shown, the pinch valve mechanism 100 has a first end 110, which includes a first projection 115, and a second end 120, which has a second projection 125. The first end 110 and second end 120 are operatively connected to each other, and as shown in the examples, in a manner which is further described below. The pinch valve mechanism 100 also includes a spring means 130 which is operatively connected to the second end 120. Notably, although the examples herein show a helical spring, it will be appreciated that any form of spring means can be used and is not limited to a helical spring.

Accordingly, one conduit 40/45 is disposed between the first end 110 and the second end 120 such that the first projection 115 and second projection 125 are biased to pinch the conduit 40/45 into a closed position, such that, compression of the spring means 130 moves the first end 110 away from the second end 120 in order to then move the conduit into an open position.

As shown in FIGS. 2 to 12, the spring means 130 can be compressed by a button 135. It will be appreciated, however, that the spring means 130 can be compressed by any type of compression means such as a lever, an actuator, or the like. In the examples shown, the pinch valve mechanism can form part of an extension of the button 135, where a pressing surface 140 of the button 135 can be external to the device 10, such that a user of the device 10 can easily press the button 135. Further, internally to the device 10, the button 135 has an extended portion 145 which is formed by the first end 110, second end 120 and further a bracket 150, which can be used to hold other conduits, such as conduit 50 of the device 10.

The usability of the button 135, which includes the feel of the button as a user compresses the button 135 to open one or more conduits 40/45, can be affected by one or more combination of variables/factors, including the spring force of the spring means 130, the shore hardness of the at least one conduit that is being compressed, the diameter of the conduit that is being compressed, and the thickness of the conduit that is being compressed.

According to one particular example, the variables have particular ranges which provides for an improved usability experience for the user of the medical device. That is, if the variables fall within the following ranges (any one or combination thereof), the feel of the button 135 as it is compressed is improved such that the useability of the medical device is improved. The ranges include:
the spring force has a range between about 5 and about 30 Newtons;
the shore hardness of the at least one tube is between about 30 and about 60 Shore A;
the diameter of the tube is between about 3 millimetres and about 15 millimetres; and;
the thickness of the tube is between about 0.2 millimetres and about 4 millimetres.

It will be appreciated that any combination of these ranges may result in the desired usability of the device.

Notably, with respect to the hardness, which is typically a measurement determined during the tube extrusion process, the variable can be substituted for a hardness of the tube measured by any other suitable means.

It will be appreciated that the usability factors can affect the way in which a user uses the medical device. That is, by improving the feel of compressing the buttons 135, this can also improve the precision of using the device. The usability factors can also improve the device by minimising the travel distance of the button 135, whilst still fully opening the valve (hence affecting the responsiveness and flow rate of the fluid within the conduit, for example), and minimise the pressure that is needed to actuate the pinch valve, whilst still being able to seal the conduits as required, and not causing the conduit to collapse under vacuum (and hence affecting the button feel).

Other factors that can also affect the button feel and usability also include the surface area of the button (that is where the user of the device will typically press to actuate the button) and the area of the valve, as further described below.

Thus, for example, if the surface area of the pressing surface 140 of the button 135 is increased (either through increasing the length or width of the button) compared to the contact area of projection 115 on the first end 110, facilitates a reduction in pressure required to be applied to the pressing surface 140 to actuate the mechanism 100, and release the conduit 40/45.

It is believed (as further described below) that the pressure applied to the conduit 40/45 is governed by the contact of area of projection 115 with the conduit and force applied by spring 130. A smaller contact area increases pressure proportionately, requiring less force to seal the conduit.

The user is thus required to provide a greater opposing force to actuate the pinch valve mechanism 100 and decompress the conduit 40/45, with the application of pressure to surface 140 to exert said force. As a larger contact area decreases pressure required proportionately, the user may comfortably apply more force to the surface.

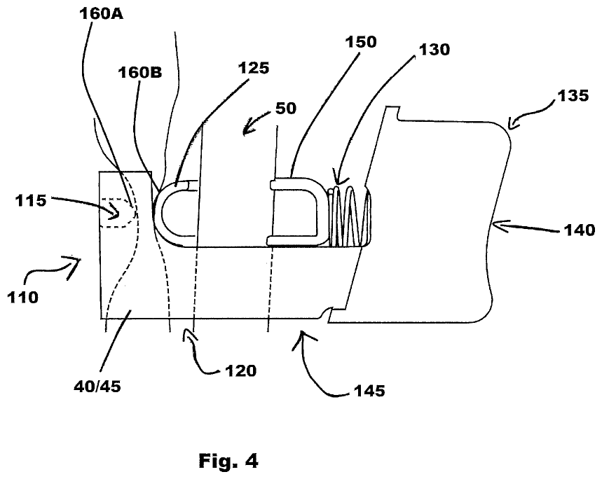

For the exemplar mechanism, a button area of 135 mm² and valve area of 9 mm² provide a 1500% reduction in pressure application under equivalent force in comparison to areas of equal size (of valve and button area). FIGS. 4 to 12 show further examples of pinch valve mechanisms with different variations of projections.

FIG. 4 shows rounded first projection 115 and second projection 125, with respective first end 160A, and second end 160B, which are rounded, where the rounded ends 160A, 160B are moved together in the biased closed state of the spring means 130, to seal the conduit 40/45 and are forced apart to open the conduit 40/45 by compression of the spring means 130.

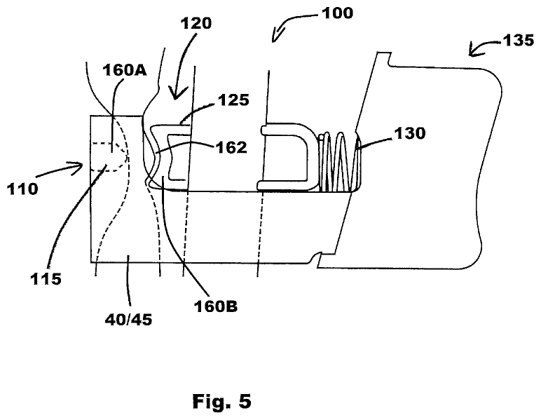

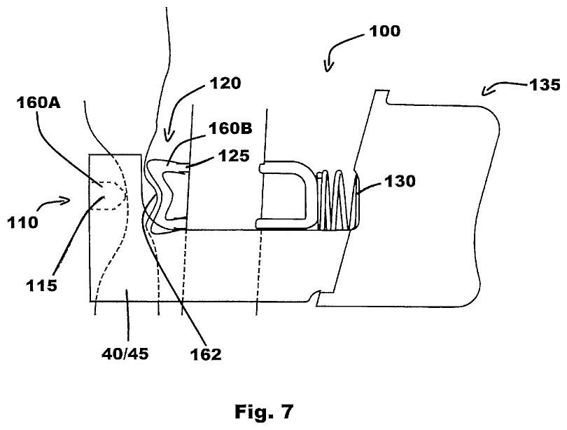

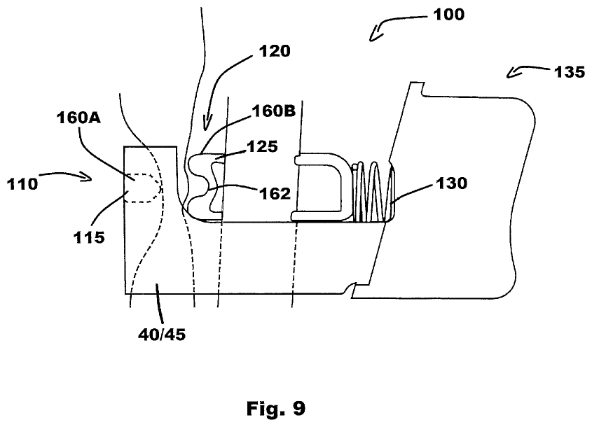

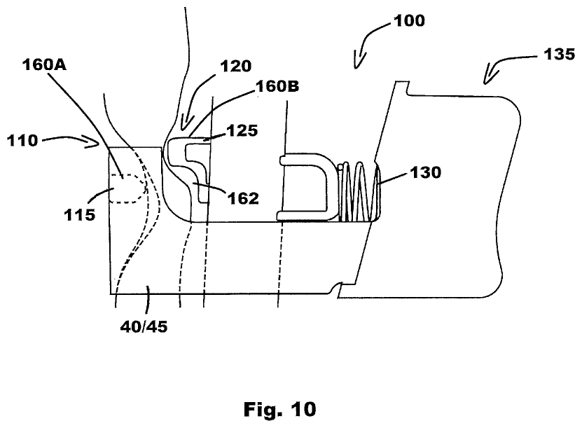

FIG. 5 shows a further example of a pinch valve mechanism 100 where the first end 160A is rounded, whereas the second end 160B has a concave portion 162 that mates with the rounded end 160A. FIG. 7 shows a similar embodiment where the concave portion 162 is slightly wider than that the second end 160B of FIG. 5. In an alternate form, FIG. 9 shows the concave portion 162 being slightly narrower in shape. And in yet a further example, FIG. 10 shows a semi-concave portion 162.

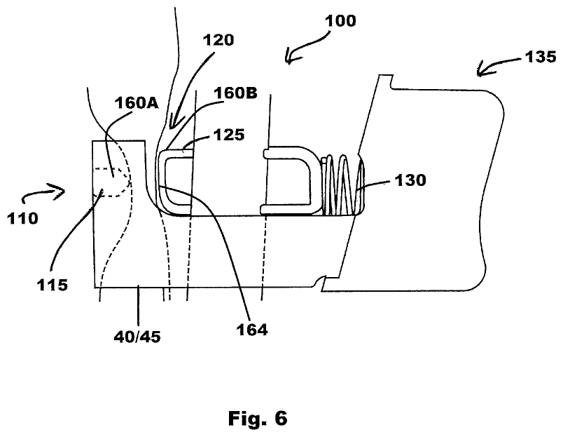

FIG. 6 shows another example of a pinch valve mechanism 100 where the first end 160A is rounded, and the second end 160B has a flatter end 164.

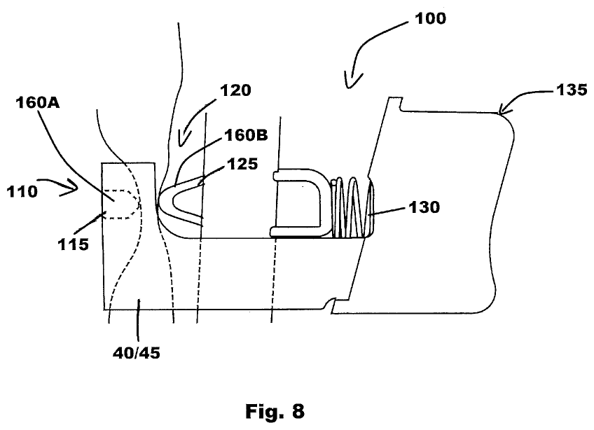

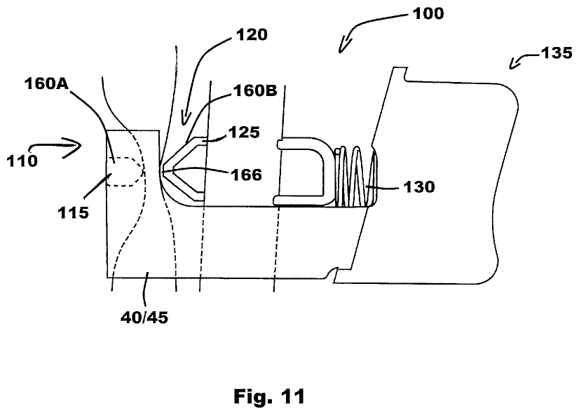

FIG. 8 shows a further example of a pinch valve mechanism 100, where the second end 160B is pointed or v-shaped. FIG. 11 also shows a v-shaped or pointed end, but with a flattened end 166.

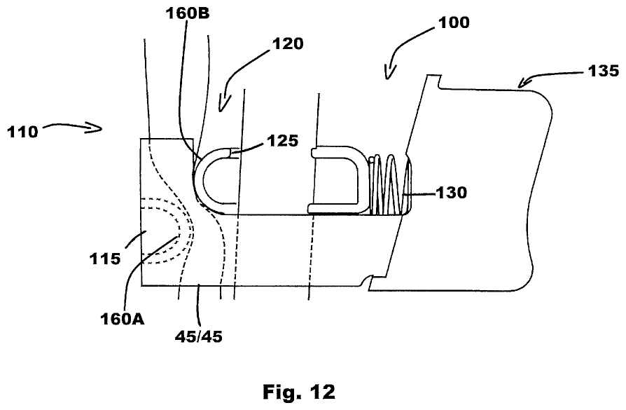

In a further example, FIG. 12 shows a pinch valve mechanism 100 where the projections 115 and 125 are slightly offset from one another.

### Further Examples

It is postulated that the theory for the above usability factors is as follows:

### Section 1

### Example Effects of the Pinch Valve Mechanism Described Herein Include:

1. Performance is equivalent to responsiveness of the button, that is, the quality of the dispensed stream and the ability to control the amount of liquid, where the desired criteria are:
   a. Responsiveness is the travel distance of the button in mm
   b. The dispensed stream should be controllable by utilising the button
   c. The stream needs to have a laminar flow and high exit velocity to reach the target site and effectively irrigate the area
2. Button feel is the minimisation of the pressure required to actuate the button to prevent fatigue
   a. This needs to be balanced with the ability to effectively seal the tube when not in use

### Section 2

### Variables

SHₜ=shore hardness of the tubing
SHₜ₍ₘᵢₙ₎=hardness below which the tubing would collapse under vacuum
Pᵥ=pressure the valve exerts on the tube
Pᵥ₍ₘᵢₙ₎=minimum pressure the valve requires to compress a tube of shore hardness SH_{T}
P_{b}=pressure the user exerts on the button
P_{b(max)}=maximum pressure the user can comfortably exert on the button
Fₛ=force the spring exerts at the specified preload
A_{b}=contact area of the button Aᵥ=contact area of the valve
Dₜ=diameter of the tube
Tₜ=wall thickness of the tube
S_{fos}=factor of safety

### Variable Value Restrictions

### Spring Force in Newtons (N)

5<Fₛ<30

Shore A of tube

30<SHₜ<60

Diameter of the tube in millimetres (mm)

3<Dₜ<15

Thickness of the tube in millimetres (mm)

0.2<Tₜ<4

### Assumptions

1. The effect of atmospheric pressure on the tube when not under vacuum due to suction is negligible
2. The spring rate and force are defined by the manufacturer and the preload by the CAD model resulting in the force specified

### Rules for Valve

The shore hardness of the tube must be greater than the minimum shore hardness required to prevent collapse under vacuum;

SH_{T}>SHₜ₍ₘᵢₙ₎

The minimum pressure the valve can exert on the tube is proportionate to shore hardness of the tube, diameter of the tube, and wall thickness of the tube;

Pᵥ₍ₘᵢₙ₎∝SH_{T}DₜTₜ

The pressure the valve exerts on the tube must be greater than the minimum pressure required for the valve to fully compress the tube;

Pᵥ<Pᵥ₍ₘᵢₙ₎

The valve pressure is equal to the force of the spring divided by the contact area of the valve;

$${P}_{v} = \frac{{F}_{s}}{{A}_{v}}$$

Therefore, the force of the spring divided by the area of the valve must be greater than the minimum pressure required to fully compress the tube;

$$\frac{{F}_{s}}{{A}_{v}} > {P}_{v ( min )}$$

Force of the spring must be greater than the product of the minimum pressure required to fully compress the tube and the area of the valve (shows the area of the valve affects the force required proportionately);

Fₛ<Pᵥ₍ₘᵢₙ₎Aᵥ

The minimum force required to compress the valve is equal to the product of the minimum valve pressure and the area of the valve;

Fᵥ₍ₘᵢₙ₎=Pᵥ₍ₘᵢₙ₎Aᵥ

Therefore, the force of the spring must be greater than the minimum force required to completely compress the tube;

Fₛ>Fᵥ₍ₘᵢₙ₎

### Rules for the Button

Similar transformations to the button;

$${P}_{b} < {P}_{b ( max )}$$

$${P}_{b} = \frac{{F}_{s}}{{A}_{b}}$$

$$\frac{{F}_{s}}{{A}_{b}} < {P}_{b ( max )}$$

$${F}_{s} < {P}_{b ( max )} {A}_{b}$$

$${F}_{b ( max )} = {P}_{b ( max )} {A}_{b}$$

$${F}_{s} < {F}_{b ( max )}$$

### Spring Force Relationship

The minimum force required to fully compress the valve is less than the spring force which is less than the maximum force the user can comfortably exert on the button;

∴Fᵥ₍ₘᵢₙ₎<Fₛ<F_{b(max)}

For the final product we need to include an engineering factor of safety in the spring force;

Fᵥ₍ₘᵢₙ₎<FₛS_{fos}<F_{b(max)}

### Section 3

### Button Pressure Relationship

The pressure that a user needs to exert on the button can be minimised by having a proportionately smaller contact area between the valve and tube, such as the projection ends, and the tube.

$${P}_{b} = \frac{{F}_{b}}{{A}_{b}} {P}_{v} = \frac{{F}_{v}}{{A}_{v}}$$

$${A}_{b} = \frac{{F}_{b}}{{P}_{b}} {A}_{v} = \frac{{F}_{v}}{{P}_{v}}$$

If the area of the button is greater than the area of the valve;

$${A}_{b} > {A}_{v}$$

$$\frac{{F}_{b}}{{P}_{b}} > \frac{{F}_{v}}{{P}_{v}}$$

If the force on the button is equal to the force experienced by the valve (as they are connected);

$${F}_{b} = {F}_{v}$$

$$\frac{{P}_{v} {F}_{b}}{{F}_{b}} > \frac{{P}_{b} {F}_{b}}{{F}_{b}}$$

Therefore, when the area of the button is greater than the area of the valve and the force is constant, the pressure on the valve is greater than the pressure on the button

Pᵥ>P_{b}

For current design measurements

$${A}_{b} \approx 135 {mm}^{2} {A}_{v} \approx 9 {mm}^{2}$$

$${P}_{b} = \frac{{F}_{bv}}{135} {P}_{v} = \frac{{F}_{bv}}{9}$$

$${F}_{bv} = 135 {P}_{b} {F}_{bv} = 9 {P}_{v}$$

$$135 {P}_{b} = 9 {P}_{v}$$

Therefore, in the given scenario the pressure exerted by the valve onto the tube is 15 times the pressure the user exerts on the button

### Section 4

### Tube Pressure

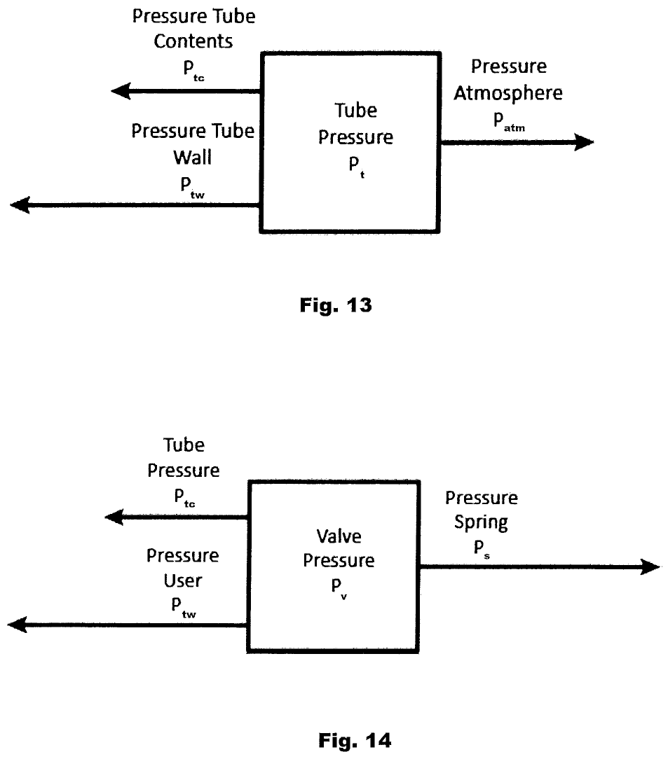

An example showing tube/conduit pressure is shown in FIG. 13, which shows the pressure exerted by the outer wall of the tube on the valve. This can be equated as follows:

Pₜ=P_{tc}−Pₐₜₘ−P_{tw}

P_{tw}>P_{tc}−Pₐₜₘ

At rest, atmospheric pressure and the pressure of the contents of the tube are equal

P_{tc}=Pₐₜₘ

Therefore, at rest pressure of the tube is equal to pressure of the tube wall

Pₜ=P_{tw}

### Valve Pressure

FIG. 14 depicts an example of the different pressures exerted by the valve mechanism.

The current valve button assembly pressure state

Pᵥ=Pₛ−P_{b}

The pressure the valve exerts on the tube is equal to the pressure the tube exerts on the valve

Pᵥ=Pₜ

as the area of the valve pressure is equal to the area of the tube pressure at their point of contact, the forces are equal

$$\frac{{F}_{v}}{{A}_{v}} = \frac{{F}_{t}}{{A}_{t}}$$

$${A}_{v} = {A}_{t}$$

$$\frac{{F}_{v} {A}_{t}}{{A}_{t}} = \frac{{F}_{t} {A}_{t}}{{A}_{t}}$$

$${F}_{v} = {F}_{t}$$

This is confirmed by Newton's third law; “When one body exerts a force on a second body, the second body simultaneously exerts a force equal in magnitude and opposite in direction on the first body”

### Button Pressure

Spring force is greater than 0

### Section 5

Kₛ=spring rate
G_{sw}=shear modulus of elasticity of the wire
d_{sw}=diameter of spring wire
Nₐₛ=number of active coils
Dₛ=mean diameter of spring

### Spring Rate and Mechanical Properties

The spring rate can be determined utilising these properties

$${K}_{s} = \frac{{G}_{sw} {d}_{sw}^{4}}{8 {N}_{as} {D}_{s}^{3}}$$

Spring Rate in Relation to Displacement

The force of the spring is defined by Hooke's law

$${K}_{s} = \frac{{F}_{s}}{{X}_{sv}} \\ {F}_{s} = {K}_{s} {X}_{sv}$$

The change in height of the spring is the free spring height minus the current height of the spring

Xₛᵥ=H_{sf}−H_{c}

The current height of the spring is the spring height when the device is at rest under preload minus the displacement of the button

H_{c}=H_{sf}−Xₚ−X_{b}

Xₛᵥ=H_{sf}−(H_{sf}−Xₚ−X_{b})

Xₛᵥ=H_{sf}−H_{sf}−Xₚ−X_{b}

Therefore, change in height is the displacement due to preload, a constant, in addition to the displacement due to movement of the button

Xₛᵥ=Xₚ−X_{b}

### Button Position

The displacement of the button valve assembly is modulated by applying pressure to the button, as the button is depressed the spring force increases according to the spring rate.

F_{b}=Fₛ−Fₜ

for any position of the valve, the force the user exerts on the button must be equal to the opposing force,
at rest, the opposing force is equal to the force of the spring minus the force from the tube

F_{b}=Fₛ−Fₜ

As the user exerts more force on the button the assembly will displace, increasing the spring force according to Hooke's law and decreasing the outward pressure of the tube until the forces in the system reaches a new equilibrium, allowing the user to govern the button position through the application of force, thus governing the flow rate through the tube.

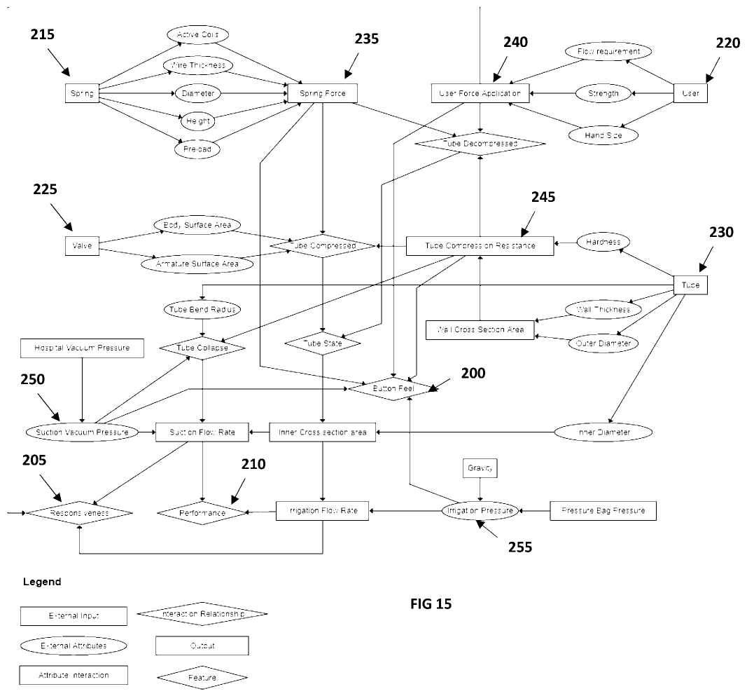

As would be appreciated by the person skilled in the art, minor characteristics of the device may be designed to suit customer requirements such as different sized handles of the device, and the like. The inter-relationship of the different characteristics of the device are shown as an example, in FIG. 15.

As shown in FIG. 15, there are three characteristics of the suction/irrigator device that can be affected by various features and inter-relationships. The characteristics include the button feel 200, responsiveness of the device 205, and performance of the device 210.

There are external and internal inputs and/or attributes that can affect each of these characteristics. As shown in FIG. 15 (and as described herein), for example, the button feel 200 can be affected by aspects of the spring 215, the user of the device 220, the valve itself 225, and the tube 230.

Thus for example, the spring 215 can be affected by aspects such as the type of coil, the wire thickness, diameter, height and preload of the spring. These can work together to produce the necessary spring force to compress the spring. The way in which the user 220 uses the device such as the flow required by the user, the strength the user uses to open/close the pinch valve, and even the hand-size of the user can possibly all affect the force which the user applies on the pinch valve mechanism. In addition to this, the valve itself at 225 can be affected by features such as the body surface area, and the armature surface area (i.e. the surface area of the first and second projections as described herein). FIG. 15 also shows, for example that the tube 230 can be affected by its hardness (referred to herein as the shore hardness), wall thickness (which is made of of the tube's outer and inner diameter).

Thus, the button feel 220 can ultimately be affected by the spring force 235, the user application force 240, the tube compression resistance 245, as well as other factors such as the suction vacuum pressure 250, and the irrigation pressure 255.

It will be appreciated by persons skilled in the art that the depiction of the relationships in FIG. 15 is an example only. Further, although it is postulated that the features shown in FIG. 15 can affect button feel (also referred to herein as usability), the features which can allow for optimal usability, as described herein, are the variables such as the spring force, shore hardness of the tube, diameter of the tube, and the wall thickness of the tube.

The term “comprise” and variants of that term such as “comprises” or “comprising” are used herein to denote the inclusion of a stated integer or integers but not to exclude any other integer or integers, unless in the context or usage an exclusive interpretation of the term is required. Those skilled in the art will appreciate that the invention described herein is susceptible to variations and modifications other than those specifically described. All such variations and modifications are to be considered within the scope and spirit of the present invention the nature of which is to be determined from the foregoing description.

1-7. (canceled)

8. A pinch valve mechanism for use in a suction/irrigator device, the device having at least one conduit for suction/irrigation, the pinch valve mechanism including:
a. a first end having a first projection;
b. a second end, operatively connected to the first end and having a second projection; and,
c. a spring means operatively connected to the second end,
wherein the at least one conduit is disposed between the first end and the second end such that the first projection and second projection are biased to pinch the at least one conduit into a closed position, and wherein, compression of the spring means moves the first end away from the second end to move the at least one conduit into an open position, and the spring means is compressed by a button; such that the usability of the button is affected by a combination of variables including at least spring force, shore hardness of the at least one conduit, diameter of the at least one conduit, and thickness of the at least one conduit.

9. The pinch valve mechanism of claim 8, wherein the spring force has a range between about 5 and about 30 Newtons.

10. The pinch valve mechanism of claim 8, wherein the shore hardness of the at least one conduit is between about 30 and about 60 Shore A.

11. The pinch valve mechanism of claim 8, wherein the diameter of the conduit is between about 3 millimetres and about 15 millimetres.

12. The pinch valve mechanism of claim 8, wherein the thickness of the at least one conduit is between about 0.2 millimetres and about 4 millimetres.

13. The pinch valve mechanism of claim 8, wherein the first and second projections have respective first and second ends, where the first and second ends are formed in different shapes to compress the at least one conduit, the shapes including any one or a combination of:
a. pointed in a V-shape;
b. an inverse V-shape;
c. curved or rounded; and,
d. flattened.

14. The pinch valve mechanism of claim 8, wherein a pressure exerted on the button is minimised by having a proportionally larger button surface area compared to a valve area.
